(19) United States
(12) Patent Application Publication
(10) Pub. No.: US 2026/0204341 A1
(43) Pub. Date: Jul. 16, 2026

(54) NON-TRANSITORY COMPUTER-READABLE RECORDING MEDIUM, CONTROL METHOD, AND INFORMATION PROCESSING APPARATUS

(71) Applicant: Fujitsu Limited, Kawasaki-shi (JP)

(72) Inventor: Fuyuka YAMADA, Kawasaki (JP)

(73) Assignee: Fujitsu Limited, Kawasaki-shi (JP)

(21) Appl. No.: 19/394,951

(22) Filed: Nov. 20, 2025

(30) Foreign Application Priority Data
Nov. 26, 2024 (JP) 2024-205663

(51) Int. Cl. G16B 5/30; G16B 15/00

(52) U.S. Cl. CPC G16B 5/30; G16B 15/00

(57) ABSTRACT

A non-transitory computer-readable recording medium has stored therein a control program that causes a computer to execute a process including determining a first parallel number of nodes to execute computation for structural information of a polymer, based on a number of atoms contained in the polymer submitting a job to a plurality of nodes, the job associating the structural information of the polymer with the first parallel number allowing a number of nodes corresponding to the first parallel number for the structural information of the polymer to execute computation for the structural information of the polymer and when a computation time of the computation for the Structural information of the polymer reaches a reference time or more, allowing the nodes that have performed the computation to update a parallel number for the polymer from the first parallel number to a second parallel number.

### CROSS-REFERENCE TO RELATED APPLICATION

This application is based upon and claims the benefit of priority of the prior Japanese Patent Application No. 2024-205663, filed on Nov. 26, 2024, the entire contents of which are incorporated herein by reference.

### FIELD

The embodiments discussed herein are related to a computer-readable recording medium and the like.

### BACKGROUND

Molecular dynamics (MD) simulations are valuable for exploring the diverse range of conformations (or shapes) that proteins can adopt.

In MD simulations, energy minimization computation is performed as pre-processing before actual simulation. Energy minimization computation is computation that searches for a structure that minimizes the protein's energy while changing the protein's structure little by little. The lower the energy of the protein, the more stable the structure.

It is noted that in order to know the structure of a large number of proteins or to predict the binding of a large number of compounds to proteins and a large number of compounds to proteins, MD simulation (energy minimization computation) is executed multiple times.

In conventional technology, when MD simulation is executed multiple times, a job is submitted to a supercomputer, and the supercomputer executes energy minimization computation. For example, frame fx of a protein is set in a job. Frame fx is information about a protein's structure at elapsed time x.

FIG. 12 is a diagram illustrating a conventional technology. The example illustrated in FIG. 12 describes a case where jobs (1) to (5) are submitted to a job scheduler 10 of a supercomputer. Job (1) includes frame f1 of protein A. Job (2) includes frame f2 of protein A. Job (3) includes frame f3 of protein A. Job (4) includes frame f4 of protein A. Job (5) includes frame f1 of protein B.

Upon accepting jobs (1) to (5), the job scheduler 10 assigns jobs (1) to (5) to each node n1 to n5 of the supercomputer in turn. For example, node n1 is assigned job (1) and executes energy minimization computation. Node n2 is assigned job (2) and executes energy minimization computation. Node n3 is assigned job (3) and executes energy minimization computation. Node n4 is assigned job (4) and executes energy minimization computation. Node n5 is assigned job (5) and executes energy minimization computation.

In FIG. 12, the length of the box for each job indicated in nodes n1 to n5 represents the time taken for the energy minimization computation. The example illustrated in FIG. 12 indicates that the computation time taken for job (5) executed by node n5 is longer than the computation time taken for jobs executed by the other nodes n1 to n4.

When the energy minimization computation by a node is completed, the job scheduler 10 assigns a new job to the node. The related technologies are described, for example, in: Japanese National Publication of International Patent Application No. 2007-508643; Japanese Laid-open Patent Publication No. 2017-37377; U.S. Patent Application Publication No. 2003/0187585; and U.S. Patent Application Publication No. 2010/0023473.

However, the conventional technology described above is unable to efficiently execute computation of protein structures.

For example, there is an upper limit to the number of jobs that can be submitted to the job scheduler 10. As explained with reference to FIG. 12, if one job is to execute energy minimization computation for a frame of one protein, a number of jobs corresponding to frames of each protein are submitted to the job scheduler 10. If the number of protein types or the number of frames is large, the number of job submissions exceeds the maximum number that can be submitted to the job scheduler 10, and efficient computation is unable to be executed.

### SUMMARY

According to an aspect of an embodiment, a non-transitory computer-readable recording medium has stored therein a control program that causes a computer to execute a process including determining a first parallel number of nodes to execute computation for structural information of a polymer, based on a number of atoms contained in the polymer submitting a job to a plurality of nodes, the job associating the structural information of the polymer with the first parallel number allowing a number of nodes corresponding to the first parallel number for the structural information of the polymer to execute computation for the structural information of the polymer and when a computation time of the computation for the Structural information of the polymer reaches a reference time or more, allowing the nodes that have performed the computation to update a parallel number for the polymer from the first parallel number to a second parallel number.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a supplemental illustration of energy minimization computation;

FIG. 2 is a diagram illustrating a first proposal;

FIG. 3 is a diagram illustrating a second proposal;

FIG. 4 is a diagram illustrating a third proposal;

FIG. 5 is a diagram illustrating an example of a data structure of an execution waiting list;

FIG. 6 is a diagram illustrating a process of an information processing apparatus according to the present embodiment;

FIG. 7 is a functional block diagram illustrating a configuration of the information processing apparatus according to the present embodiment;

FIG. 8 is a diagram illustrating an example of a data structure of protein basic information;

FIG. 9 is a diagram illustrating an example of the computation results of the parallel number after update;

FIG. 10 is a flowchart illustrating a process procedure of the information processing apparatus according to the present embodiment;

FIG. 11 is a diagram illustrating an example of a hardware configuration of a computer that implements the same functions as the information processing apparatus of the present embodiment; and

FIG. 12 is a diagram illustrating a conventional technology.

### DESCRIPTION OF EMBODIMENT(S)

Preferred embodiments of the present invention will be explained with reference to accompanying drawings. It is noted that the present invention is not limited by the embodiments.

Before describing the present embodiment, the protein energy minimization computation will be explained. The initial structure of a protein may have high energy. The high energy of the protein indicates that the protein's structure is unstable. Energy minimization computation is performed to stabilize the protein's structure.

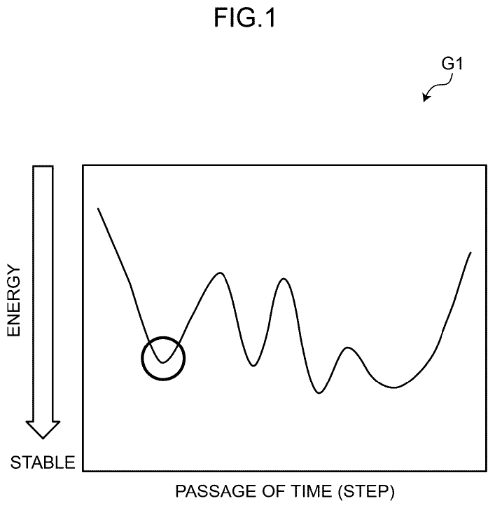

FIG. 1 is a supplemental illustration of the energy minimization computation. The vertical axis of graph G1 illustrated in FIG. 1 corresponds to energy, and the horizontal axis corresponds to the passage of time. In the energy minimization computation, the energy is computed while updating the coordinates of atoms of a protein, and a local minimum of energy is searched for. The steepest descent method, conjugate gradient method, and the like are used as energy minimization algorithms.

Various computations are performed on the result of the energy minimization computation (protein structure with minimized energy), including production runs and training of a high-dimensional neural network potential (HDNNP), which is one of machine learning potentials to estimate energy from structural information.

First, second, and third proposals for efficiently executing computation of protein structures will now be described in turn.

First, the first proposal will be described. Since there is an upper limit to the number of jobs that can be submitted to the job scheduler 10, the first proposal reduces the number of jobs by setting a plurality of frames of a protein in one job.

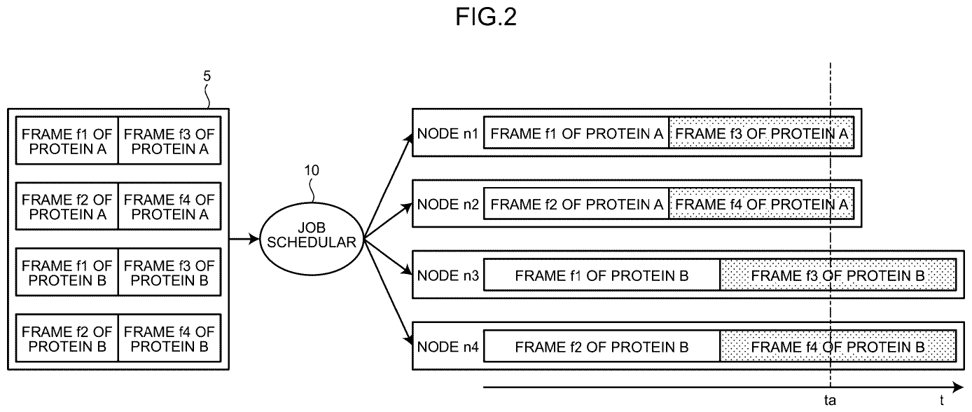

FIG. 2 is a diagram illustrating the first proposal. In the first proposal, a job list 5 is generated and submitted to the job scheduler 10 such that a plurality of frames of a protein are set in one job. For example, in the job list 5, frames f1 and f3 of protein A, frames f2 and f4 of protein A, frames f1 and f3 of protein B, and frames f2 and f4 of protein B are set.

For example, the job scheduler 10 assigns frames f1 and f3 of protein A to node n1 and assigns frames f2 and f4 of protein A to node n2. The job scheduler 10 assigns frames f1 and f3 of protein B to node n3 and assigns frames f2 and f4 of protein B to node n4.

As illustrated in FIG. 2, in the first proposal, the job list 5 in which a plurality of frames of a protein are set in one job is submitted to the job scheduler 10 to allow the nodes to execute multiple energy minimization computations, thereby reducing the number of jobs. This process solves the problem that the number of job submissions exceeds the maximum number that can be submitted to the job scheduler 10 if the number of protein types or the number of frames is large.

Here, in addition to the condition that there is a limit to the maximum number of jobs that can be submitted to the job scheduler 10, there is also a condition that there is an upper limit to the execution time of the jobs that are executed at each node. When the upper limit of execution time is reached during job execution, the energy minimization computation is aborted in the middle.

In FIG. 2, the length of the protein frame indicated in nodes n1 to n4 is the time taken for the energy minimization computation, and the upper limit of execution time is time ta. Then, at node n1, the energy minimization computation for frame f3 of protein A is aborted in the middle. At node n2, the energy minimization computation for frame f4 of protein A is aborted in the middle. At node n3, the energy minimization computation for frame f3 of protein B is aborted in the middle. At node n4, the energy minimization computation for frame f4 of protein B is aborted in the middle.

In other words, the first proposal has the problem that the energy minimization computation is aborted in the middle when the upper limit of execution time is reached during job execution.

The second proposal and the third proposal are proposals that solve the problem of the first proposal.

The second proposal will be described. In addition to the first proposal, the second proposal determines the parallel number of nodes in advance, according to the scale of proteins, so that more energy minimization computations can be completed in one job.

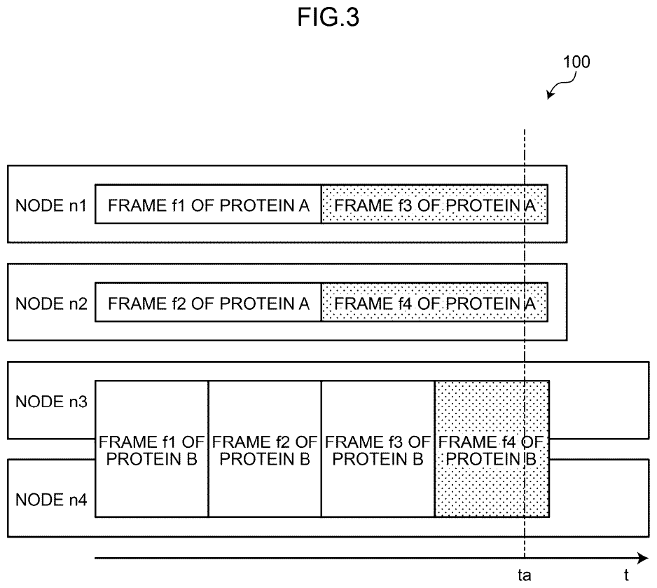

FIG. 3 is a diagram illustrating the second proposal. Protein A and protein B will be used here in the description. The parallel number determined in advance according to the scale of protein A is “1”, and the parallel number determined in advance according to the scale of protein B is “2”. An apparatus that executes the process of the second proposal is denoted as “information processing apparatus 100”. The specific method of calculating the parallel number will be described later.

The parallel number for protein A is “1”. Thus, the information processing apparatus 100 assigns frames f1 and f3 of protein A to node n1. The information processing apparatus 100 assigns frames f2 and f4 of protein A to node n2.

The parallel number for protein B is “2”. Thus, the information processing apparatus 100 assigns frames f1, f2, f3, and f4 of protein B to nodes n3 and n4.

In FIG. 3, the length of the protein frame indicated in nodes n1 to n4 is the time taken for the energy minimization computation, and the upper limit of execution time is time ta. Then, at node n1, the energy minimization computation for frame f3 of protein A is aborted in the middle. At node n2, the energy minimization computation for frame f4 of protein A is aborted in the middle. At nodes n3 and n4, the energy minimization computation for frame f4 of protein B is aborted in the middle.

The job assignment according to the first proposal described with reference to FIG. 2 is compared with the job assignment according to the second proposal described with reference to FIG. 3. Then, in the first proposal, the energy minimization computation for four frames is aborted in the middle, whereas in the second proposal, the number of frames for which the energy minimization computation is aborted is reduced to three.

In other words, compared with the first proposal, the second proposal can reduce the number of frames for which the energy minimization computation is aborted in the middle, while reducing the number of jobs.

The third proposal will now be described. In the second proposal above, the parallel number of nodes is determined in advance according to the scale of proteins, but in the third proposal, the parallel number of nodes is changed dynamically. In the third proposal, if the energy minimization computation for one frame of a protein that is executed on a node exceeds a reference time, the parallel number of nodes for the corresponding protein is increased. The reference time is set in advance.

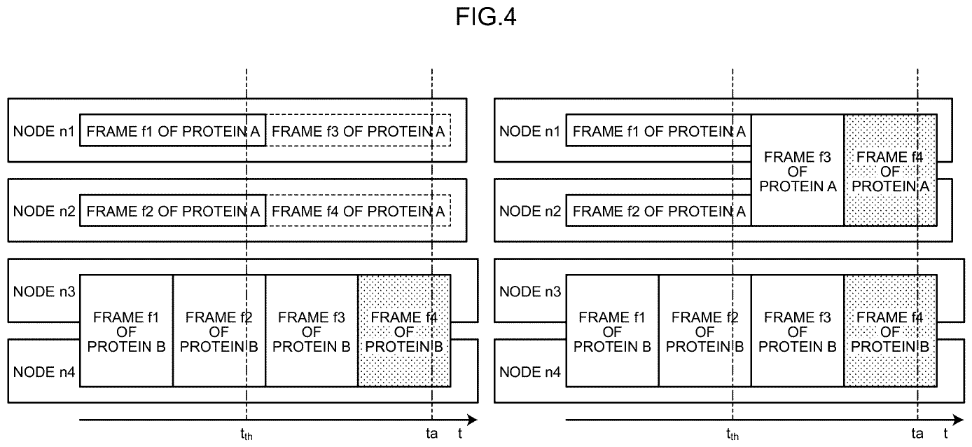

FIG. 4 is a diagram illustrating the third proposal. Protein A and protein B will be used here in the description. The parallel number according to the scale of protein A determined in advance is “1”, and the parallel number according to the scale of protein B is “2”. An apparatus that executes the process of the third proposal is denoted as “information processing apparatus 100”.

The parallel number for protein A is “1”. Thus, the information processing apparatus 100 assigns frame f1 of protein A to node n1. The information processing apparatus 100 assigns frame f2 of protein A to node n2. The information processing apparatus 100 updates the parallel number for protein A to “2” because the time of energy minimization computation for frame f1 of protein A by node n1 and for frame f2 of protein A for node n2 reaches a reference time tₜₕ or more.

For frames f3 and subsequent frames of protein A, the information processing apparatus 100 assigns frames with the parallel number for protein A as “2”. For example, the information processing apparatus assigns frames f3 and f4 of protein A to nodes n1 and n2.

The parallel number for protein B is “2”. Thus, the information processing apparatus 100 assigns frames f1, f2, f3, and f4 of protein B to nodes n3 and n4. The information processing apparatus 100 keeps the parallel number for protein B at “2” because the energy minimization computation for frame f1 of protein B for node n3 is less than the reference time tₜₕ.

In FIG. 4, the length of the protein frame indicated in nodes n1 to n4 is the time taken for the energy minimization computation, and the upper limit of execution time is time ta. Then, at nodes n1 and n2, the energy minimization computation for frame f4 of protein A is aborted in the middle. At nodes n3 and n4, the energy minimization computation for frame f4 of protein B is aborted in the middle.

The job assignment according to the second proposal described with reference to FIG. 3 is compared with the job assignment according to the third proposal described with reference to FIG. 4. Then, in the second proposal, the energy minimization computation for three frames is aborted in the middle, whereas in the third proposal, the number of frames for which the energy minimization computation is aborted is reduced to two.

The third proposal can reduce the number of frames for which the energy minimization computation is aborted in the middle more than the second proposal, while reducing the number of jobs in the same way as the second proposal.

The process of the information processing apparatus 100 according to the present embodiment will now be described more specifically. Basically, the information processing apparatus 100 executes the process based on the third proposal.

As a precondition, a reference number of atoms of protein (Threshold Atoms) is “1000”. The reference time tₜₕ is “4 h”. The upper limit of execution time ta is “9 h”. The time taken for energy minimization computation is “½” when the number of nodes used is doubled. The systems (number of atoms) of proteins A, B, and C are as follows.
System of protein A=900
System of protein B=1200
System of protein C=500

The information processing apparatus 100 identifies the parallel number to assign to each protein from the number of atoms of proteins A, B, and C. For example, the information processing apparatus 100 calculates a division value (rounded down to the nearest whole number) by dividing the number of atoms of a protein by Threshold Atoms, and determines the value obtained by adding one to the division value as the parallel number for the protein. As the result, the parallel numbers for proteins A, B, and C are as follows.
Protein A=1+900/1000≈1
Protein B=1+1200/1000≈2
Protein C=1+500/1000≈1

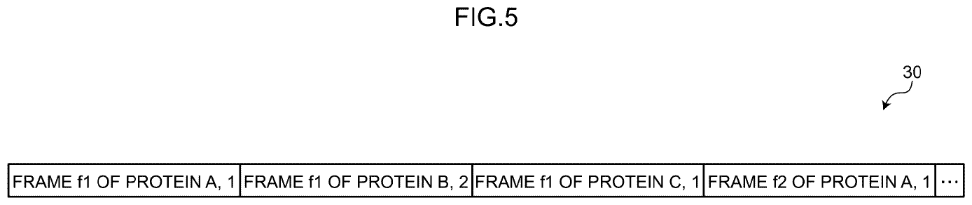

The information processing apparatus 100 creates an execution waiting list. FIG. 5 is a diagram illustrating an example of a data structure of the execution waiting list. For example, the information processing apparatus 100 arranges proteins A, B, and C frame by frame, starting from the top of an execution waiting list 30, and thereafter the arrangement is random. The protein frames set in the execution waiting list 30 are set together with the parallel number. For example, “frame f1 of protein A, 1” indicates that the parallel number for protein A is “1”. “Frame f1 of protein B, 2” indicates that the parallel number for protein B is “2”. “Frame f1 of protein C, 1” indicates that the parallel number for protein C is “1”.

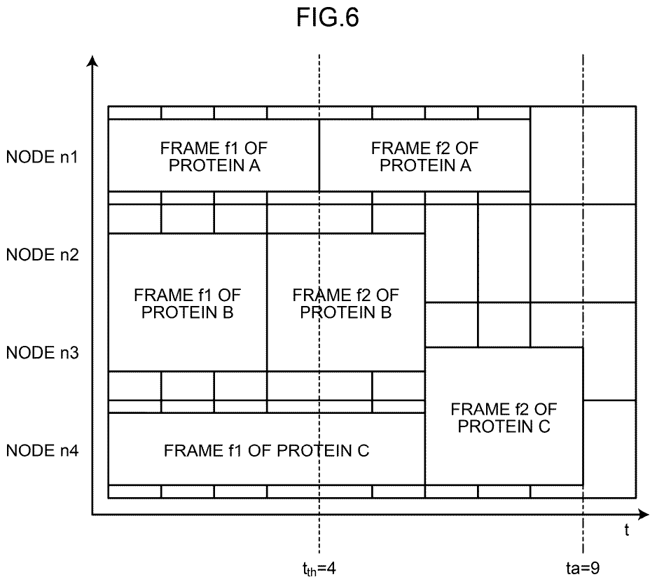

FIG. 6 is a diagram illustrating the process of the information processing apparatus according to the present embodiment. The information processing apparatus 100 submits jobs to nodes n1 to n4 in order from the top of the execution waiting list 30.

The information processing apparatus 100 submits frame f1 of protein A to node n1. The information processing apparatus 100 submits frame f1 of protein B to nodes n2 and n3. The information processing apparatus 100 submits frame f1 of protein C to node n4. For example, the completion time of the energy minimization computation for each protein frame is the following time.
Execution time for frame f1 of protein A: 4 h
Execution time for frame f1 of protein B: 3 h
Execution time for frame f1 of protein C: 6 h

In the information processing apparatus 100, among the above execution times, the execution time for frame f1 of protein C is 6h, which is more than the reference time t_{th.} In this case, the information processing apparatus 100 updates the parallel number for protein C to “2”. For example, the information processing apparatus 100 updates the parallel number for frames of protein C after frame f1 of protein C in the execution waiting list 30 to 2.

The information processing apparatus 100 then submits frame f2 of protein B to nodes n2 and n3. The information processing apparatus 100 submits frame f2 of protein A to node n1. The information processing apparatus 100 submits frame f2 of protein C to nodes n3 and n4 at the timing when two or more nodes become free.

Nodes n1 to n4 terminate the job when the upper limit of execution time ta is reached. In the example illustrated in FIG. 6, the process ends without the energy minimization computation for each protein frame being aborted in the middle.

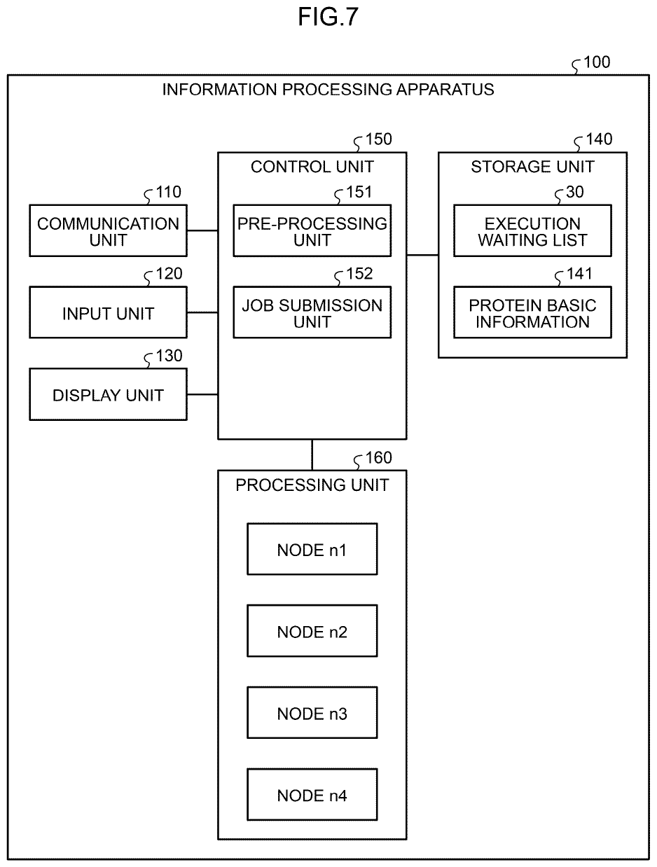

Next, a configuration example of the information processing apparatus 100 that executes the process described with reference to FIG. 6 will be described. FIG. 7 is a functional block diagram illustrating a configuration of the information processing apparatus according to the present embodiment. As illustrated in FIG. 7, the information processing apparatus 100 includes a communication unit 110, an input unit 120, a display unit 130, a storage unit 140, a control unit 150, and a processing unit 160.

The communication unit 110 executes data communication with an external device and the like via a network. The communication unit 110 is a network interface card (NIC) or the like. For example, the communication unit 110 may obtain protein basic information 141 from an external device or the like.

The input unit 120 is an input device that inputs various information to the control unit 150 of the information processing apparatus 100. For example, the input unit 120 corresponds to a keyboard, a mouse, a touch panel, and the like. For example, the user may operate the input unit 120 to input the reference number of atoms of protein (Threshold Atoms) and the reference time tₜₕ.

The display unit 130 is a display device that displays information output from the control unit 150.

The storage unit 140 has the execution waiting list 30 and the protein basic information 141. The storage unit 140 is a memory or the like.

Frames of each protein are set in the execution waiting list 30. The other description for the execution waiting list 30 is the same as the description provided with reference to FIG. 5.

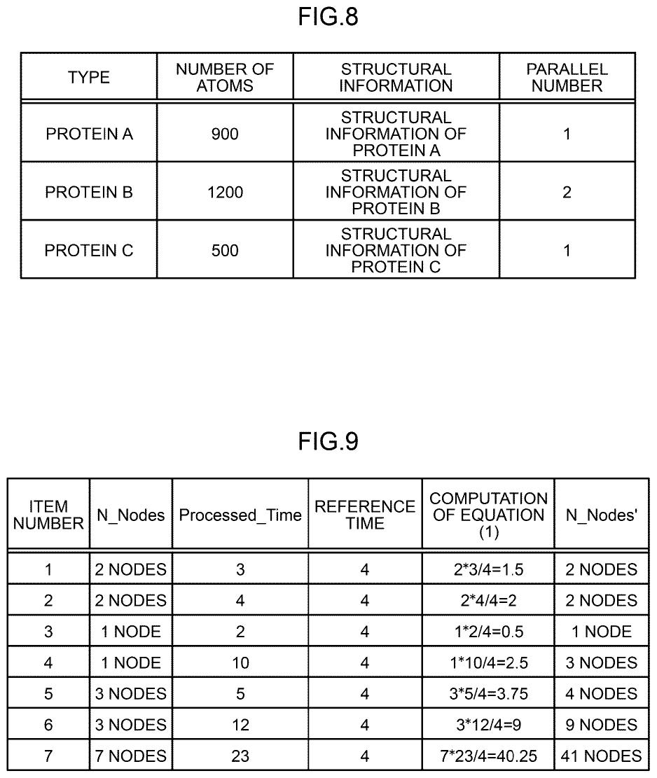

The protein basic information 141 holds various information about proteins. FIG. 8 is a diagram illustrating an example of a data structure of the protein basic information. As illustrated in FIG. 8, the protein basic information 141 includes type, number of atoms, structural information, and parallel number. The type is the type of protein. The number of atoms is the number of atoms in the corresponding protein. The structural information is the structural information of the corresponding protein. For example, the structural information includes time-series frames. The parallel number is the parallel number computed in advance.

The control unit 150 will now be described. The control unit 150 includes a pre-processing unit 151 and a job submission unit 152. The control unit 150 is a central processing unit (CPU), a graphics processing unit (GPU), or the like.

The pre-processing unit 151 determines the parallel number for each protein, based on the reference number of atoms of protein (Threshold Atoms) specified in advance and the number of atoms in the protein basic information 141. The pre-processing unit 151 registers the determined parallel number in the protein basic information 141.

The pre-processing unit 151 obtains a frame for each protein from the protein basic information 141 and generates the execution waiting list 30. The pre-processing unit 151 sets the parallel number in the frame of the protein according to the type of protein. The pre-processing unit 151 registers the execution waiting list 30 in the storage unit 140.

The job submission unit 152 is a processing unit that submits the execution waiting list 30 as jobs to the processing unit 160.

The processing unit 160 includes nodes n1 to n4. Nodes n1 to n4 are CPUs or the like. Upon accepting a job submission, nodes n1 to n4 obtain one frame of a protein from the execution waiting list 30 and execute energy minimization computation for the obtained frame.

It is noted that in nodes n1 to n4, when the parallel number for the corresponding protein is 1, one node executes energy minimization computation for a frame of the protein. In nodes n1 to n4, when the parallel number for the corresponding protein is 2, two nodes execute energy minimization computation for the same protein frame.

When the energy minimization computation is completed, nodes n1 to n4 output the computation results to the control unit 150 and the like.

When the time taken for the energy minimization computation reaches the reference time tₜₕ or more, nodes n1 to n4 access the execution waiting list 30 and add 1 to the parallel number for the corresponding protein frame. Nodes n1 to n4 abort the energy minimization computation when the time taken for the energy minimization computation reaches the upper limit of execution time ta or more.

For example, a node may update the parallel number (N_Nodes) for a protein as in Equation (1) when the time taken for minimization computation (Processed_Time) for frame fx of the protein reaches the reference time tₜₕ or more. The parallel number after update is N_Nodes′. However, the numerical value calculated by Equation (1) is rounded up to the nearest whole number.

$${N\_Nodes}^{′} = N\_Nodes \times Processed\_Time / reference time {t}_{t h} \qquad ( 1 )$$

For example, if a node has executed frame f1 of protein A and has frames f2 to f100 remaining, the node updates the parallel number (N_Node) of the remaining frames f2 to f100 with N_Node′ after update.

Nodes n1 to n4 repeat the above process until the execution waiting list 30 is empty. In the example illustrated in FIG. 7, the processing unit 160 includes nodes n1 to n4, but the processing unit 160 may further include other nodes.

FIG. 9 is a diagram illustrating an example of the computation results of the parallel number after update. FIG. 9 associates item number, N_Nodes, Processed_Time, reference time, computation of Equation (1), and N_Nodes′. The item number is the number that distinguishes each row. N_Nodes is the parallel number before update. Processed_Time is the time taken for the energy minimization computation. The reference time is the reference time t_{th.} The computation of Equation (1) is the result of substituting N_Nodes, Processed_Time, and the reference time into Equation (1). N_Nodes′is the parallel number after update.

For example, in item number 1, N_Nodes=2,Processed_Time=3, and the reference time=4, which are substituted into Equation (1), resulting in the parallel number after update N_Nodes′=2. The description for the other items 2 to 7 is omitted.

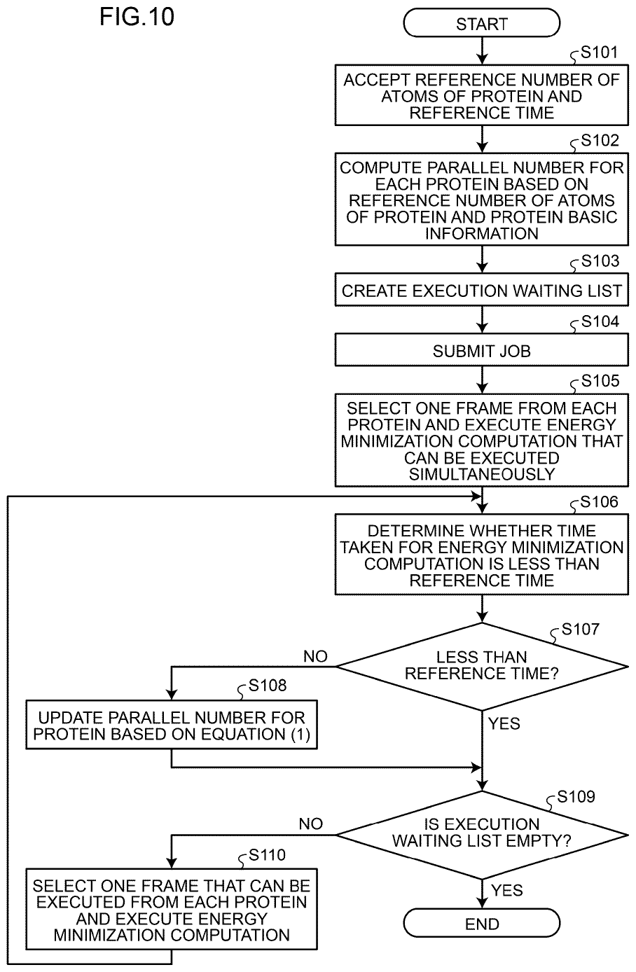

Next, an example of a processing procedure of the information processing apparatus 100 according to the present embodiment will be described. FIG. 10 is a flowchart illustrating a process procedure of the information processing apparatus according to the present embodiments. As illustrated in FIG. 10, the information processing apparatus 100 accepts the reference number of atoms of protein (Threshold Atoms) and the reference time tₜₕ (step S101).

The pre-processing unit 151 of the information processing apparatus 100 computes the parallel number for each protein based on the reference number of atoms of protein and the protein basic information 141 (step S102). The pre-processing unit 151 creates the execution waiting list 30 (step S103).

The job submission unit 152 submits jobs (execution waiting list 30) to the processing unit 160 (step S104). Each node of the processing unit 160 selects one frame from each protein in the execution waiting list 30 and executes as many energy minimization computations as can be executed simultaneously (step S105).

Each node determines whether the time taken for the energy minimization computation is less than the reference time (step S106). Each node moves to step S109 if the time taken for the energy minimization computation is less than the reference time (Yes at step S107).

On the other hand, if the time taken for the energy minimization computation is not less than the reference time (No at step S107), each node updates the parallel number for the protein based on Equation (1) (step S108) and moves to step S109.

Each node terminates the process if the execution waiting list 30 is empty (Yes at step S109). On the other hand, if the execution waiting list 30 is not empty (No at step S109), the node (free node) selects one frame that can be executed from the protein from the execution waiting list 30, executes the energy minimization computation (step S110), and moves to step S106.

Next, the effects of the information processing apparatus 100 according to the present embodiment will be described. The control unit 150 of the information processing apparatus 100 determines the parallel number of nodes to execute computation for a frame of a protein based on the number of atoms contained in the protein, creates the execution waiting list 30, and submits the execution waiting list 30 to the processing unit 160. In the processing unit 160, a number of nodes corresponding to the parallel number execute computation for the frame of the protein. When the computation time reaches the reference time or more, the nodes update the parallel number for the protein. With this configuration, the computation of protein structures can be executed efficiently.

For example, according to the information processing apparatus 100, the energy minimization computation for each protein frame can be completed as explained with reference to FIG. 6 and the like.

The information processing apparatus 100 calculates the parallel number after update based on the division value obtained by dividing the computation time by the reference time and the parallel number before update. With this configuration, the appropriate parallel number can be updated dynamically according to the scale (number of atoms) of the protein system.

The information processing apparatus 100 determines the parallel number of nodes based on the result of dividing the number of atoms contained in the protein by the reference number of atoms. With this configuration, the parallel number can be set in advance according to the scale of proteins even when the time taken for energy minimization computation is unknown.

The information processing apparatus 100 generates the execution waiting list 30, and each node obtains a frame (structural information) of a protein in turn from the execution waiting list 30 and executes energy minimization computation. With this configuration, each node can execute energy minimization computation appropriately.

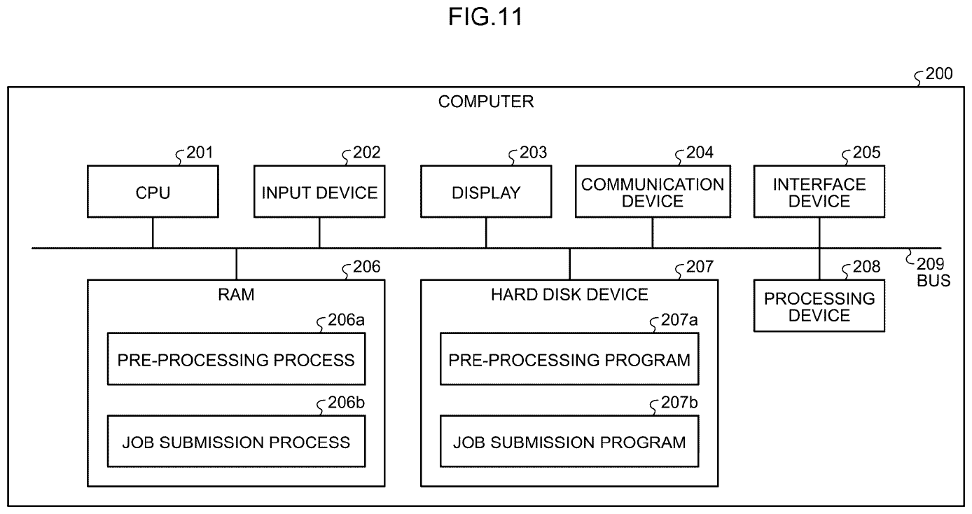

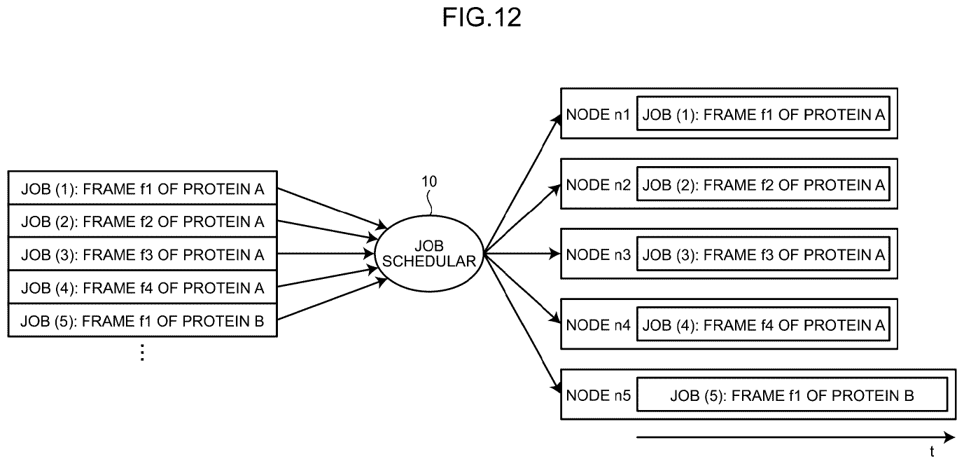

Next, an example of a hardware configuration of a computer that implements the same functions as the information processing apparatus 100 described above will be described. FIG. 11 is a diagram illustrating an example of a hardware configuration of a computer that implements the same functions as the information processing apparatus of the present embodiment.

As illustrated in FIG. 11, a computer 200 includes a CPU 201 that executes various arithmetic operations, an input device 202 that accepts data input from a user, and a display 203. The computer 200 also includes a communication device 204 that communicates data with an external device and the like via a wired or wireless network, and an interface device 205. The computer 200 also includes a RAM 206 that temporarily stores various information, a hard disk device 207, and a processing device 208. Each device 201 to 208 is connected to a bus 209.

For example, the processing device 208 corresponds to the processing unit 160 illustrated in FIG. 7 and includes a plurality of nodes. Each of the nodes is a CPU or the like.

The hard disk device 207 includes a pre-processing program 207a and a job submission program 207b. The CPU 201 reads each of the programs 207a and 207b and loads the program in the RAM 206.

The pre-processing program 207a functions as a pre-processing process 206a. The job submission program 207b functions as a job submission process 206b.

The processing of the pre-processing process 206a corresponds to the processing of the pre-processing unit 151. The processing of the job submission process 206b corresponds to the processing of the job submission unit 152.

Each of the programs 207a and 207b is not necessarily stored in the hard disk device 207 from the beginning. For example, each program is stored on a “portable physical medium” such as flexible disk (FD), CD-ROM, DVD, magneto-optical disk, or IC card that is inserted into the computer 200. The computer 200 may then read and execute each of the programs 207a and 207b.

Computation of protein structures can be executed efficiently.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiment(s) of the present invention has(have) been described in detail, it should be understood that the various changes, substitutions, and alterations could be made hereto without departing from the spirit and scope of the invention.

What is claimed is

1. A non-transitory computer-readable recording medium having stored therein a control program that causes a computer to execute a process comprising:
determining a first parallel number of nodes to execute computation for structural information of a polymer, based on a number of atoms contained in the polymer;
submitting a job to a plurality of nodes, the job associating the structural information of the polymer with the first parallel number;
allowing a number of nodes corresponding to the first parallel number for the structural information of the polymer to execute computation for the structural information of the polymer; and
when a computation time of the computation for the Structural information of the polymer reaches a reference time or more, allowing the nodes that have performed the computation to update a parallel number for the polymer from the first parallel number to a second parallel number.

2. The non-transitory computer-readable recording medium according to claim 1, wherein the process further includes calculating the second parallel number after update, based on a division value obtained by dividing the computation time by the reference time and the first parallel number before update.

3. The non-transitory computer-readable recording medium according to claim 1, wherein the process further includes determining the first parallel number of the nodes, based on a result of dividing a number of atoms contained in the polymer by a reference number of atoms.

4. The non-transitory computer-readable recording medium according to claim 1, wherein the job includes structural information of a plurality of proteins, and the process further includes allowing the nodes to obtain the structural information of the proteins from the job in turn to execute the computation.

5. A control method comprising:
determining a first parallel number of nodes to execute computation for structural information of a polymer, based on a number of atoms contained in the polymer;
submitting a job to a plurality of nodes, the job associating the structural information of the polymer with the first parallel number;
allowing a number of nodes corresponding to the first parallel number for the structural information of the polymer to execute computation for the structural information of the polymer; and
when a computation time of the computation for the Structural information of the polymer reaches a reference time or more, allowing the nodes that have performed the computation to update a parallel number for the polymer from the first parallel number to a second parallel number, by using a computer.

6. The control method according to claim 5, further including calculating the second parallel number after update, based on a division value obtained by dividing the computation time by the reference time and the first parallel number before update.

7. The control method according to claim 5, further including determining the first parallel number of the nodes, based on a result of dividing a number of atoms contained in the polymer by a reference number of atoms.

8. The control method according to claim 5, wherein the job includes structural information of a plurality of proteins, and the control method further includes allowing the nodes to obtain the structural information of the proteins from the job in turn to execute the computation.

9. An information processing apparatus comprising:
a memory; and
a processor coupled to the memory and configured to:
determine a first parallel number of nodes to execute computation for structural information of a polymer, based on a number of atoms contained in the polymer;
submit a job to a plurality of nodes, the job associating the structural information of the polymer with the first parallel number;
allow a number of nodes corresponding to the first parallel number for the structural information of the polymer to execute computation for the structural information of the polymer; and
when a computation time of the computation for the Structural information of the polymer reaches a reference time or more, allow the nodes that have performed the computation to update a parallel number for the polymer from the first parallel number to a second parallel number.

10. The information processing apparatus according to claim 9, wherein the processor is further configured to calculate the second parallel number after update, based on a division value obtained by dividing the computation time by the reference time and the first parallel number before update.

11. The information processing apparatus according to claim 9, wherein the processor is further configured to determine the first parallel number of the nodes, based on a result of dividing a number of atoms contained in the protein by a reference number of atoms.

12. The information processing apparatus according to claim 10, wherein the job includes structural information of a plurality of proteins, and the processor is further configured to allow the nodes to obtain the structural information of the proteins from the job in turn to execute the computation.
